(19) United States
(12) Patent Application Publication
(10) Pub. No.: US 2025/0195416 A1
(43) Pub. Date: Jun. 19, 2025

(54) DISSOLVABLE POLYMERIC OCULAR INSERT FOR DRUGS DELIVERY

(71) Applicant: Alcon Inc., Fribourg (CH)

(72) Inventors: Qiaozhi Lu, Fort Worth, TX (US); Hongzhu Liao, Arlington, TX (US); Adam C. Reboul, Roswell, GA (US); Ye Hong, Flower Mound, TX (US); Randall Renato Kolega, Arlington, TX (US); Cécile Adrienne Maissa, Fort Worth, TX (US)

(21) Appl. No.: 18/978,032

(22) Filed: Dec. 12, 2024

Related U.S. Application Data
(60) Provisional application No. 63/610,074, filed on Dec. 14, 2023.

(51) Int. Cl. A61K 9/00; A61K 9/06; A61K 31/335; A61K 47/10; A61K 47/32; A61K 47/36; A61K 47/38

(52) U.S. Cl. CPC A61K 9/0051; A61K 9/06; A61K 31/335; A61K 47/10; A61K 47/32; A61K 47/36; A61K 47/38

(57) ABSTRACT

An ocular insert, comprising: (a) at least one first mucoadhesive polymer selected from the group consisting of hyaluronic acid or sodium hyaluronate, polyvinylpyrrolidone, carboxyl methyl cellulose, and a combination thereof; (b) at least one second mucoadhesive polymer selected from the group consisting of hydroxyethyl guar, hydroxypropyl guar (HP-guar), hydroxypropyl methylcellulose (HPMC), and a combination thereof; (c) at least one plasticizer and (d) olopatadine in an amount from 2% to 20% by weight of the ocular insert, wherein the ocular insert has no visible recrystallization under examination with a polarized light microscope at approx. 100× magnification, wherein the ocular insert has a single-layer, homogeneous structure, wherein the at least one first mucoadhesive polymer and the at least one second mucoadhesive polymer are together present in the ocular insert in an amount of from about 75% to about 90% by weight of the ocular insert.

### FIELD OF THE DISCLOSURE

The present disclosure generally relates to a dissolvable polymeric ocular insert for placement on the outer surface (conjunctiva) of the eye to deliver Olopatadine. The dissolvable ocular insert is a polymeric film of specific dimensions to be placed in the inferior conjunctival fornix and the polymeric film comprises of at least two mucoadhesive polymers. The dissolvable polymeric film releases mucoadhesive polymers and olopatadine into the eye for an extended duration of time compared to topical drop dosage forms.

### BACKGROUND

Providing and maintaining adequate concentrations of bioactive agents, such as drugs, for example, in the pre-corneal tear film for extended periods of time is one of the major problems plaguing methods and systems for ocular drug delivery. When they are applied as eye drops, most drugs retained poorly on the cornea. Drainage of instilled drug with the tear fluid leads to a short duration of action. The additional pre-corneal factors that contribute to the poor ocular bioavailability of many drugs when instilled in the eye as drops are tear turnover and drug binding to tear fluid proteins.

Topical delivery via eye drops that account for majority of all ophthalmic formulations is very inefficient. Only very small amount of the drug applied as drops is retained on the corneal surface and reaches the ocular tissue, while the rest is lost due to tear drainage. The drug mixes with the tear fluid upon instillation and has a short residence time and only small amount of the drug gets absorbed and the remaining flows through the upper and the lower canaliculi into the lacrimal sac.

In recent years, a wide variety of research has been carried out to develop ocular inserts useful as a dosage form for treating a variety of eye disorders. There have been many attempts to construct ocular inserts for delivering a drug over a prolonged period of time, generally hours or days to perhaps months.

LACRISERT® inserts are commercially available erodible ocular inserts that are used to treat dry eye. LACRISERT® insert is a sterile, translucent, solid rod which measures 1.27 mm in diameter and 3.5 mm in length and is made of hydroxypropyl cellulose. For administration, it is placed into the inferior cul-de-sac of the eye beneath the base of the tarsus by a patient or a medical practitioner.

However, there also are challenges in using these types of inserts. For example, LACRISERT® inserts tend to dissolve slowly and can remain in the eye even after 15-20 hours. The rod is hard and inelastic with edges due to rod-shaped design. The slow dissolving properties coupled with the rod hardness and design may lead to side effects including blurred vision, foreign body sensation and/or discomfort, ocular irritation or hyperemia, hypersensitivity, photophobia, eyelid edema, and caking or drying of viscous material on eyelashes. The most common side effect of these hydroxypropyl cellulose ophthalmic inserts is blurred vision due to the long retention time of the insert. Thus, additional approaches are needed to develop polymeric eye inserts that are comfortable and improve patient compliance.

Olopatadine ophthalmic (eye) drops is used to treat itching and redness of the eye due to allergic conjunctivitis, caused by pollen, ragweed, grass, animal hair, or dander. It stabilizes mast cells and blocks histamines that have already been released from attaching to the histamine (H1) receptors in the eye, breaking the chain of allergic reaction. Pataday® allergy relief eye drops contain olopatadine HCl as the active ingredient, and are available over the counter without a prescription: Pataday® Twice daily 0.1% ophthalmic solution, Pataday® once daily 0.2% ophthalmic solution, and Pataday® once daily extra strength 0.7% ophthalmic solution. These products are all in the solution dosage form. Furthermore, ocular allergy is often accompanied with dry eye symptoms. Thus, there is a need for new Olopatadine delivery systems that increase the residence time of the Olopatadine in the eye, thereby reducing wastage, to treat both itching of the eye and dry eye.

### SUMMARY

The invention provides an ocular insert, comprising: (a) at least one first mucoadhesive polymer selected from the group consisting of hyaluronic acid or sodium hyaluronate, polyvinylpyrrolidone, carboxyl methyl cellulose, and a combination thereof; (b) at least one second mucoadhesive polymer selected from the group consisting of hydroxyethyl guar, hydroxypropyl guar (HP-guar), hydroxypropyl methylcellulose (HPMC), and a combination thereof; (c) at least one plasticizer and (d) Olopatadine in an amount from 2% to 20% by weight of the ocular insert, wherein the ocular insert has no visible recrystallization under examination with a microscope with polarizing filter at approx. 100× magnification, wherein the at least one first mucoadhesive polymer and the at least one second mucoadhesive polymer are together present in the ocular insert in an amount of from about 75% to about 90% by weight of the ocular insert.

### BRIEF DESCRIPTION OF THE DRAWINGS

For a more complete understanding of this disclosure, reference is now made to the following description, taken in conjunction with the accompanying drawings, in which:

FIG. 1A-1B illustrate in vitro release of Olopatadine (OLO) and HA in water (1A) and PBS (1B) in different dissolution media.

FIG. 2 illustrates release of API from gel films with different compositions in PBS.

### DETAILED DESCRIPTION

Unless defined otherwise, all technical and scientific terms used herein have the same meaning as commonly understood by one of ordinary skill in the art to which this invention belongs. Generally, the nomenclature used herein, and the laboratory procedures are well known and commonly employed in the art. Conventional methods are used for these procedures, such as those provided in the art and various general references. Where a term is provided in the singular, the inventors also contemplate the plural of that term. The nomenclature used herein, and the laboratory procedures described below are those well-known and commonly employed in the art.

“About” as used herein means that a number referred to as “about” comprises the recited number plus or minus 1-10% of that recited number.

“Optional” or “optionally” means that the subsequently described event or circumstance can or cannot occur, and that the description includes instances where the event or circumstance occurs and instances where it does not.

As used in this application, the term “ocular insert” refers to a sterile, thin solid or semisolid (gel) article that is placed into the cul-de-sac or conjuctival sac of an eye when being used by a patient and optionally impregnated with a drug.

The term “soluble”, in reference to a compound or material in a solvent, means that the compound or material can be dissolved in the solvent to give a solution with a concentration of at least about 0.1% by weight at room temperature (i.e., from about 20° C. to about 30° C.).

The term “insoluble”, in reference to a compound or material in a solvent, means that the compound or material can be dissolved in the solvent to give a solution with a concentration of less than 0.005% by weight at room temperature (as defined above).

A “mucoadhesive polymer” refers to a polymer capable of bind to a mucus or mucous membrane that adheres to epithelial surfaces (e.g., the gastrointestinal tract, the lung, the eye, etc.), as known to a person skilled in the art. It should point out that mucoadhesive polymers have been widely described in the literature. See, for example, the article entitled “Mucoadhesive Drug Delivery System: A Review” by Dharmendra et al. in Int. J. Pharm. Biol. Arch. 2012, 3(6):1287-1291 and the article entitled “Polymers in Mucoadhesive Drug-Delivery System: A Brief Note” published by Roy et al. in Designed Monomers and Polymers 2009, 12(6):483-495.

The invention is partly based on the discovery that eye allergy & dry eye symptom co-morbidities are more frustrating and difficult to achieve good relief for both. Currently there is no treatment/product that addresses symptom relief of both allergies and dry eyes. Olopatadine containing dissolvable polymeric ocular insert provides day-long relief of both dry eye and allergy symptoms. Prolonged delivery of lubricating polymers and allergy relief ingredient directly to the ocular surface.

The invention is also partly based on the discovery that Olopatadine can be loaded into dissolvable polymeric ocular insert up to 20% w/w without visible recrystallization under examination with a microscope with polarizing filter at approx. 100× magnification. Olopatadine is known to form polymorphism. Since polymorphism and crystallization are related, these two questions are addressed together. Polymorphism is the condition when a solid chemical exists in more than one crystalline form (polymorphs). Polymorphs of a given chemical may have very different physical properties and bioavailability. Ocular gel films with olopatadine were prepared by solubilizing components in water first, followed by drying to remove water. During the drying process, olopatadine may crystalize into one of its polymorphs, resulted in different solubility and dissolution rates, which may eventually affect the bioavailability. FDA and ICH regulations also require that if polymorphs exist in a product, they need to be fully characterized. Therefore, we want to avoid crystallization of olopatadine during gel film preparation and keep it in its amorphous form along with other components. Marta Łaszczet. Structural and Physicochemical Studies of Olopatadine Hydrochloride Conformational Polymorphs Journal of Pharmaceutical Sciences 105 (2016) 2419-2426.

In this application, olopatadine is a known compound that can be obtained by the methods disclosed in U.S. Pat. No. 5,116,863, the entire contents of which are hereby incorporated by reference in the present specification. Olopatadine, have anti-allergic and anti-inflammatory activity. Olopatadine is the cis form of the compound having the formula:

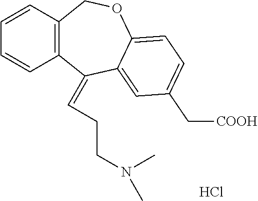 [SMILES: CN(C)CC/C=C1/c2ccccc2COc2ccc(CC(=O)O)cc21 ; Cl]

Chemical Name of Olopatadineis 11-[(Z)-3-(Dimethylamino)propylidene]-6-11-dihydrodibenz[b,e] oxepin-2-acetic acid hydrochloride. Olopatadine is an inhibitor of the release of histamine from the mast cell and a relatively selective histamine H1-antagonist that inhibits the in vivo and in vitro type 1 immediate hypersensitivity reaction including inhibition of histamine induced effects on human conjunctival epithelial cells. Olopatadine is devoid of effects on alpha-adrenergic, dopamine and muscarinic type 1 and 2 receptors. Olopatadine is used to relieve signs and symptoms of allergic conjunctivitis (an allergic reaction affecting the eyes), such as itchy eyes, red eyes, and swelling of the surface of the eye. Olopatadine works by blocking histamine, a substance released by the body during an allergic reaction.

The invention is partly based on the discovery that Olopatadine can be loaded into dissolvable polymeric ocular insert up to 20% w/w without visible recrystallization under examination with a polarized light microscope at approx. 100× magnification.

The biomaterial for forming a dissolvable polymeric ocular insert according to embodiments of the present disclosure comprises: (a) at least one first mucoadhesive polymer selected from the group consisting of hyaluronic acid or sodium hyaluronate, polyvinylpyrrolidone, carboxyl methyl cellulose, and a combination thereof; (b) at least one second mucoadhesive polymer selected from the group consisting of hydroxyethyl guar, hydroxypropyl guar (HP-guar), hydroxypropyl methylcellulose (HPMC), and a combination thereof.

According to the present application, the at least one first mucoadhesive polymer and the at least one second mucoadhesive polymer are together present in the ocular insert in an amount of from about 75% to about 90% by weight of the ocular insert.

The preferred biocompatible polymers are hyaluronic acid, guar and derivatives and/or combinations thereof. Hyaluronic acid is an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. As used herein, the term hyaluronic acid also includes salt forms of hyaluronic acid such as sodium hyaluronate. A preferred hyaluronic acid is sodium hyaluronate. The weight average molecular weight of the hyaluronic acid used in insert of the present invention may vary but is typically weight average of 0.75 to 5.0 M Daltons. In one embodiment, the HA has a weight average molecular weight of 0.75 to 4 M Daltons. In another embodiment, the HA has a weight average molecular weight of 1 to 4 M Daltons.

The galactomannans of the present invention may be obtained from numerous sources. Such sources include from fenugreek gum, guar gum, locust bean gum and tara gum. Additionally, the galactomannans may also be obtained by classical synthetic routes or may be obtained by chemical modification of naturally occurring galactomannans. As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-.beta.-D-mannopyranosyl units with. Alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 is most preferred. Additionally, other chemically modified variations of the polysaccharides are also included in the “galactomannan” definition. For example, hydroxyethyl, hydroxypropyl and carboxymethyl hydroxypropyl substitutions may be made to the galactomannans of the present invention. Non-ionic variations to the galactomannans, such as those containing alkoxy and alkyl (C1-C6) groups are particularly preferred when a soft gel is desired (e.g., hydroxypropyl substitutions). Substitutions in the non-cis hydroxyl positions are most preferred. An example of non-ionic substitution of a galactomannan of the present invention is hydroxypropyl guar, with a molar substitution of about 0.4. Anionic substitutions may also be made to the galactomannans. Anionic substitution is particularly preferred when strongly responsive gels are desired, Preferred galactomannans of the present invention are guar and hydroxypropyl guar. Hydroxypropyl guar is particularly preferred. The weight average molecular weight of the Hydroxypropyl guar in the dissolvable medical device of the present invention may vary, but is typically 1 to 5 M Daltons. In one embodiment, the Hydroxypropyl guar has a weight average molecular weight of 2 to 4 M Daltons. In another embodiment, the Hydroxypropyl guar has a weight average molecular weight of 3 to 4 M Daltons.

Polymers used in dissolvable polymeric ocular insert according to embodiments of the present disclosure should be non-toxic and able to solubilize in eye fluids to ensure that the insert is eventually cleared from the eye, generally within 15 to 120-minute time frame. It should be appreciated that the polymer(s) selected should be mucoadhesive. It also should be appreciated that at least two polymers may be blended according to embodiments of the present disclosure. For example, in an embodiment of the present disclosure, hyaluronic acid (HA) may be blended with tamarind seed polysaccharide (TSP) because TSP has been shown to increase residence time of HA in aggregate blends and the blend has desired film mechanical and lubrication properties. In other embodiments of the present disclosure, as described in further detail below, hyaluronic acid may be combined with HP guar.

In some embodiments of the present disclosure, the preferred biocompatible polymers also include polyvinyl pyrrolidine (PVP). PVP is also a mucoadhesive polymer. The weight average molecular weight of the PVP in the polymeric film of the present invention may vary but is typically 4,000 Dalton to 3 M Daltons. In one embodiment, the PVP has a weight average molecular weight of 40 K Daltons to 2 M Daltons. In another embodiment, the PVP has a weight average molecular weight of 0.5 M Daltons to 2 M Daltons.

In some embodiments of the present disclosure, the preferred biocompatible polymers also include a non-ionic cellulose derivative. Any kind of non-ionic cellulose derivative can be used in this application, for example, hydroxypropyl methyl cellulose (“HPMC”), hydroxypropyl cellulose (“HPC”), methyl cellulose (MC), hydroxyethyl cellulose (HEC), methyl hydroxyethyl cellulose (MHEC) and methyl hydroxypropyl cellulose (MHPC), hydroxyethyl methyl cellulose (HEMC), although other non-ionic cellulose derivatives can be used in the present invention. These non-ionic cellulose derivatives can be used alone or any mixture thereof. Of these non-ionic cellulose derivatives, MC, HPMC, and HEMC having a methoxy content in the molecule of 20% to 30% by weight. According to the present application, hydroxypropyl methyl cellulose (“HPMC”) is a preferable non-ionic cellulose derivative. The hydroxypropyl methylcellulose may have a weight average molecular weight from 400 K to 3 M Daltons, from 600 K to 2 M Daltons, from 700 K to 2 M Daltons, or from 800 K to 1 M Daltons.

In some embodiments, the non-ionic cellulose derivative (for example HPMC) is present in amount of from about 5% to about 50%, about 10% to about 40%, about 15% to about 35%, about 20% to about 30%; the one or more mucoadhesive polymers are present in an amount of from about 40% to about 80% w/w, about 50% to about 75% w/w, about 55% to about 75% w/w, or about 60% to about 70% w/w of the polymeric eye insert, provided that the sum of the % w/w of all components in an ocular insert is 100% w/w.

In some embodiments of the present disclosure, the preferred biocompatible polymers also include carboxymethyl cellulose (CMC) or cellulose gum. CMC is cellulose derivative with carboxymethyl groups (—CH2-COOH) bound to some of the hydroxyl groups of the glucopyranose monomers that make up the cellulose backbone. It is often used as its sodium salt, sodium carboxymethyl cellulose. The carboxymethyl cellulose has a degree of carboxymethyl substitution from 0.5 to 0.9 and a molecular weight from 100,000 Da to 300,000 Da. Examples of carboxymethylcellulose polymers are Carboxymethyl cellulose commercially sold by CPKelko as Finnfix®GDA, hydrophobically modified carboxymethyl cellulose, for example the alkyl ketene dimer derivative of carboxymethylcellulose sold commercially by CPKelco as Finnfix®SH1, or the blocky carboxymethylcellulose sold commercially by CPKelco as Finnfix®V.

In some embodiments, the carboxymethyl cellulose is present in amount of from about 5% to about 50%, about 10% to about 40%, about 15% to about 35%, about 20% to about 30%; the one or more mucoadhesive polymers are present in an amount of from about 40% to about 80% w/w, about 50% to about 75% w/w, about 55% to about 75% w/w, or about 60% to about 70% w/w of the polymeric eye insert, provided that the sum of the % w/w of all components in an ocular insert is 100% w/w.

In some embodiments of the present disclosure, a softener and/or plasticizer may be added to the one or more polymers to facilitate fabrication of a softer, malleable delivery system and also provide improved comfort in covering the cornea. A plasticizer may soften the material to provide for desirable dissolution rates. It should be appreciated softeners and/or plasticizers may be low or high-molecular weight compounds, including not limited to, polyethylene glycol (PEG) and derivatives thereof, water, Vitamin E, and triethyl citrate. The weight average molecular weight of the PEG in the polymeric film of the present invention may vary but is typically 200 Dalton to 100,000 Daltons. In one embodiment, the PEG has a weight average molecular weight of 200 to 12000 Daltons. In another embodiment, the PEG has a weight average molecular weight of 200 to 6000 Daltons.

In some embodiments, the HP guar is present in an amount of from about 5% to about 60% w/w, preferably 15% to about 50% w/w, more preferably 25% to about 40 w/w by dry weight of the polymeric film. The PVP is present in an amount of from about 1% to about 30% w/w, preferably 5% to about 25% w/w, more preferably 10% to about 20 w/w by dry weight of the polymeric film. The hyaluronic acid (HA) is present in an amount of from about 5% to about 60% w/w, preferably 15% to about 50% w/w, more preferably 25% to about 40 w/w by dry weight of the polymeric film. The HPMC is present in an amount of from about 5% to about 60% w/w, preferably 15% to about 50% w/w, more preferably 25% to about 40 w/w by dry weight of the polymeric film. The PEG is present in an amount of from about 1% to about 30% w/w, preferably 5% to about 25% w/w, more preferably 10% to about 20 w/w by dry weight of the polymeric film. According to the present application, the total amount of ingredients of the polymeric dissolvable ocular insert is equal to 100% w/w.

The overall dry weight or mass of the polymeric film may be in the range of about 1 to about 12 mg, or about 2 to about 10 mg, and in particular embodiments may be from about 2.5 to about 8 mg.

In some embodiments, the polymeric film has a thickness of about 30-300 μm, about 30-250 μm, about 30-150 μm, about 30-120 μm, preferably about 30-50.

In some embodiments, the polymeric film has circular shape about 2 mm to 13 mm in diameter or other shapes have the same area corresponding to circular shape about 2 mm to 13 mm in diameter. In still some embodiments, the polymeric film has a contact lens shape and prefers about 11 mm to 13 mm in diameter.

In another aspect, the invention provides a method for producing an ocular insert, comprising the step of:
(1) obtaining a solution for casting an ocular insert that comprises:
(a) at least one first mucoadhesive polymer selected from the group consisting of hyaluronic acid or sodium hyaluronate, polyvinylpyrrolidone, carboxyl methyl cellulose, and a combination thereof;
(b) at least one second mucoadhesive polymer selected from the group consisting of hydroxyethyl guar, hydroxypropyl guar (HP-guar), hydroxypropyl methylcellulose (HPMC), and a combination thereof;
(c) at least one plasticizer and
(d) olopatadine in an amount from 2% to 20% by weight of the ocular insert, wherein the at least one first mucoadhesive polymer and the at least one second mucoadhesive polymer are together present in the ocular insert in an amount of from about 75% to about 90% by weight of the ocular insert; and
(2) solution casting the solution in a mold to obtain the ocular insert.

In still a further aspect, the invention provides a method for using of an ocular insert of the invention (as described above) for treating a dry eye and eye allergy disease or disorder of an eye of a subject, comprising administering the ocular insert into the eye.

In some embodiments, an ocular insert has a single-layer, homogeneous structure. In other embodiments, an ocular insert has a multilayered structure and comprises a first polymer layer sandwiched between two second polymer layers, wherein the first polymer layer has a higher concentration of olopatadine than two second polymer layers. With such a layered structure, an ocular insert of the invention can have a well-controlled disintegration kinetics for each layer, i.e., the outer polymer layers can be disintegrated (dissolved) first whereas the inner polymer layer can be disintegrated (dissolved) later. Such an ocular insert can offer more flexibility to tune properties of selected layers to achieve desired overall olopatadine dissolution profile.

Polymeric film according to embodiments of the present disclosure may be made using various processing techniques, including but not limited to, compression molding and solution casting.

The polymeric film of the present disclosure is a platform to deliver pharmaceutically active agents (for example, olopatadine) to treat ocular surface disease, before, during or post eye surgery. In some embodiments, the polymeric film can be used to prolong exposure of pharmaceutically active agents or provide extended drug delivery of pharmaceutically active agents to the eye. Thus, in some embodiments, the present disclosure provides a method of providing extended drug delivery or prolonging exposure of a pharmaceutically active agent to the eye, by administering a polymeric film including the pharmaceutically active agent (drugs) to a patient in need thereof. The drug is added in the film during manufacturing the film or onto the film after the film is placed on the outer surface of the eye or onto the film after manufacturing but before placing on the outer surface of the eye.

The following non-limiting Examples are provided to illustrate embodiments of the invention.

### EXAMPLES

Procedure below on how to manufacture and cast corneal shields. Slight variations in volume casted and drying times based on corneal shield thickness (I to III). Target thickness is ˜120 microns).

### Example I

Procedure to make 30-50 μm thickness (dry film) ocular inserts with olopatadine:

Part 1: Compounding of polymeric solutions with olopatadine. Various compositions are defined in Table 1. A total concentration of approx. 0.75-1.00% may be prepared.

For example: to make 0.75% w/w solution, approx. 750 mL of purified water is added to glass bottle, set up on a over-head mixer. HP Guar, PVP and HPMC is added to water while mixing at approx. 400 rpm. After mixing for approx. 30 min, the polymer solution is steam-sterilized at 121° C. for 20 min. A 1.0% stock solution of olopatadine HCl is prepared, and appropriate amount is added to polymer solution, followed by addition of HA and PEG. The mixture is stirred for approx. 1 hour or until the viscous solution is homogenous. More purified water is added to Q.S. to desired total weight. The solution may be vacuum degassed to remove bubbles.

Part 2: Ocular insert film casting. Square glass bottom molds (5×5 in) are utilized here, and approx. 80 to 140 g of polymer solution is poured into the mold. Solution is dried in heated oven (approx. 45° C.) until dry films are formed. To make the films easier to process, the bulk films may be placed in humidity chamber (25° C., 40% RH) to equilibrate. Individual ocular insert films are cut out by a 4- or 6-mm biopsy punch. The final ocular insert film has a water content of approx. 1-15%.

### Example 2

Ocular allergy is often accompanied with dry eye symptoms, therefore incorporating antihistamines in polymeric gel films eliminates the need of applying separate treatments for eye allergy and dry eye.

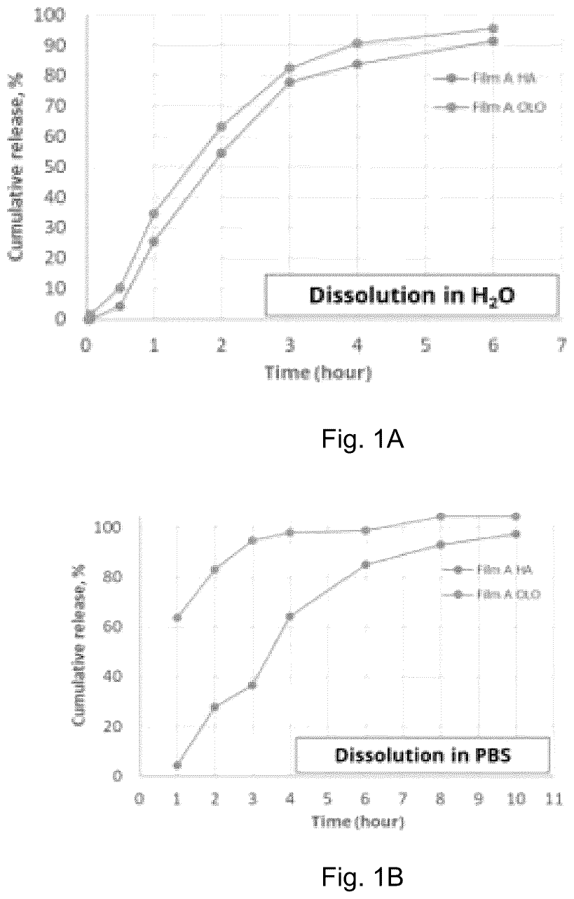

Olopatadine is a dual-action antihistamine, the active pharmaceutical ingredient (API) in Pataday anti-allergy eye drops. Polymer gel films containing olopatadine (API) were produced by adding the API in polymer solution prior to film casting and drying. A series of formulations based on the original Ontake gel film (formulation code OTF1) have been demonstrated in the laboratory. Hydrophilic polymers with more hydrophobic domains such as hydroxypropyl methylcellulose (HPMC) has been utilized to control dissolution (Table 1). Up to approx. 10-15% API can be incorporated in polymer gel films without crystallization of the API (FIG. 1), as olopatadine is known to form polymorphism. When API loading in gel film is increased to approx. 20%, crystalline olopatadine was observed under a polarized light microscope.

TABLE 1
Gel films with olopatadine: composition examples

| | Original | | | | |
|---|---|---|---|---|---|
| Sample # | gel film | A | B | C | D |
| Formulation | OTF1 | AG1 | AG2 | AG11 | AG12 |
| Code | | | | | |
| HP Guar | 40% | 38% | 36% | 40% | — |
| HPMC | — | — | — | 40% | 40% |
| K100M | | | | | |
| HA EP2.4 | 40% | 38% | 36% | — | 40% |
| PVP K90 | 10% | 9% | 9% | — | — |
| PEG400 | 10% | 10% | 9% | 10% | 10% |
| Olopatadine | — | 5% | 10% | 10% | 10% |
| Handleability | Soft | Soft | Soft | Brittle | Soft |
| Transparency | Slightly | Slightly | Slightly | Opaque | Transparent |
| | Opaque | Opaque | Opaque | | |

### Example 3

Release of olopatadine from polymer gel films was studied in vitro using water or phosphate buffered saline (PBS) as the dissolution media. Dissolution was performed using USP Apparatus 7 and samples were collected by its autosampler. HPLC—with UV-vis detector was used to measure component concentration during dissolution samples at different time points. It was noted that olopatadine release is faster in PBS than in water (FIGS. 1A and 1B). Olopatadine HCl remains as a zwitterion at physiological pH due to the negatively charged carboxylic group and positively charged tertiary amino group on the molecule, therefore possible mechanism of API release from the gel films into dissolution media is through ion exchange. HA in gel films may complex with olopatadine through ionic interaction. When gel films are placed in PBS, cationic ions such as sodium and potassium can compete with olopatadine to bind with sodium hyaluronate, hence accelerating the dissociation of API with polymers in the gel films.

FIGS. 1A and 1B illustrate that no burst release of olopatadine occurred. Dissolution media plays a main role in the release profiles, as ionic bonding occurred between HA and Olopatadine. Ionic polymer provides some prolong release. Diffusion is believed to be the main release mechanism. PBS: phosphate-buffered saline pH 7.2; DI water: deionized water.

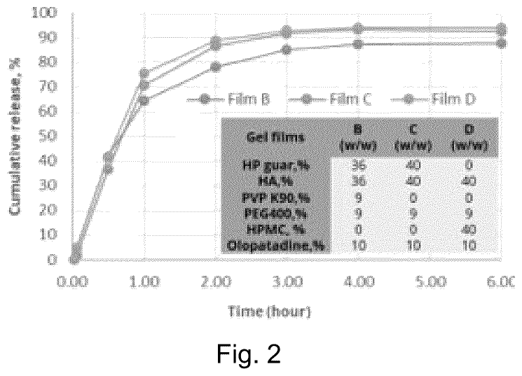

This hypothesis was further confirmed using formulations containing different amount of HA (FIG. 2). Film type C and D have a more sustained released of API compared to film type B because of the increase HA composition. No crystallization and no burse release were observed in Film B-D, after loading 10% of active. Based on non-cumulative dissolution profiles, both Film C and D are more prolonged release of API than Film B. However, film C is rigid and appears to be opaque. Overall, film D showed most sustained release profile of API, without signs of recrystallization and is softer than other prototypes.

### Example 4

The polymeric ocular inserts can be utilized as a delivery vehicle for both ocular lubricants/demulcents and antihistamines, thereby providing symptomatic relief for patients with both dry eye disease and ocular allergy. By loading the API at 5-20% level, ocular inserts can provide comparable dosing to Pataday™ anti-allergy eye drops (Table 2). In addition, the prolonged release of the polymer and API components, as demonstrated in the above in-vitro studies, may provide significantly longer ocular relief than eye drops which are quickly removed from the ocular surface.

TABLE 2
API dose calculation of Pataday eye drops and gel films

| | Pataday ™ | Paraday ™ | |
|---|---|---|---|
| | once a | (extra | Polymeric |
| | day | strength) | ocular |
| Product | (16 hours) | (24 hours) | inserts |
| Olopatadine (%) | 0.20% | 0.70% | 5-20% |
| Dose/drop size | 30 | 30 | 2.4 |
| (mg) | | | |
| Total API per dose | 0.08 | 0.21 | 0.12-0.48 |
| (mg) | | | |

1) An ocular insert, comprising:
(a) at least one first mucoadhesive polymer selected from the group consisting of hyaluronic acid or sodium hyaluronate, polyvinylpyrrolidone, carboxyl methyl cellulose, and a combination thereof;
(b) at least one second mucoadhesive polymer selected from the group consisting of hydroxyethyl guar, hydroxypropyl guar (HP-guar), hydroxypropyl methylcellulose (HPMC), and a combination thereof;
(c) at least one plasticizer and
(d) olopatadine in an amount from 2% to 20% by weight of the ocular insert, wherein the ocular insert has no visible recrystallization under examination of with an polarized light microscope at approx. 100× magnification, wherein the at least one first mucoadhesive polymer and the at least one second mucoadhesive polymer are together present in the ocular insert in an amount of from about 75% to about 90% by weight of the ocular insert.

2) The dissolvable ocular insert of claim 1, wherein the olopatadine is present in an amount from 5% to 15% by weight of the ocular insert.

3) The dissolvable ocular insert of claim 2, wherein the Olopatadine is present in an amount from 8% to 12% by weight of the ocular insert.

4) The dissolvable ocular insert of claim 3, wherein the Olopatadine is present in an amount from 9% to 10% by weight of the ocular insert.

5) The dissolvable ocular insert of claim 1, wherein the at least one first mucoadhesive polymer is selected from the group consisting of hyaluronic acid or sodium hyaluronate, polyvinylpyrrolidone and combinations thereof.

6) The dissolvable ocular insert of claim 5, wherein the at least one first mucoadhesive polymer is hyaluronic acid or sodium hyaluronate.

7) The dissolvable ocular insert of claim 1 wherein the at least one second mucoadhesive polymer are HP guar, HPMC and combinations thereof.

8) The dissolvable ocular insert of claim 7, wherein the at least one second mucoadhesive polymer is HPMC.

9) The ocular insert of claim 1, wherein the at least one first mucoadhesive polymer and the at least one second mucoadhesive polymer are together present in the ocular insert in an amount of from about 80% to about 90% by weight of the ocular insert.

10) The dissolvable ocular insert of claim 1, wherein the plasticizer or softener is selected from the group consisting of: polyethylene glycol (PEG), a PEG derivative, water, Vitamin E, glycerin and triethyl citrate.

11) The dissolvable ocular insert of claim 10, wherein the plasticizer or softener is present in an amount of from about 2% to about 30% w/w, about 5% to about 25% w/w, about 5% to about 20% w/w, or about 5% to about 15% w/w of the polymeric film by dry weight of the polymeric film, provided that the sum of the % w/w of all components in an ocular insert is 100% w/w.

12) The dissolvable ocular insert of claim 10, wherein the plasticizer or softener is PEG.

13) The dissolvable ocular insert of claim 1, wherein the insert is comprised of about 40% HPMC, about 10% Olopatadine, about 40% sodium hyaluronate, about 10% PEG.

14) A method for producing an ocular insert, comprising the step of:
(1) obtaining a solution for casting an ocular insert that comprises:
(a) at least one first mucoadhesive polymer selected from the group consisting of hyaluronic acid or sodium hyaluronate, polyvinylpyrrolidone, carboxyl methyl cellulose, and a combination thereof;
(b) at least one second mucoadhesive polymer selected from the group consisting of hydroxyethyl guar, hydroxypropyl guar (HP-guar), hydroxypropyl methylcellulose (HPMC), and a combination thereof;
(c) at least one plasticizer and
(d) olopatadine in an amount from 2% to 20% by weight of the ocular insert,
wherein the at least one first mucoadhesive polymer and the at least one second mucoadhesive polymer are together present in the ocular insert in an amount of from about 75% to about 90% by weight of the ocular insert; and
(2) solution casting the solution in a mold to obtain the ocular insert.
